(19) United States
(12) Patent Application Publication
(10) Pub. No.: US 2005/0124557 A1
(43) Pub. Date: Jun. 9, 2005

(54) Use of benzothiazepines having activity as inhibitors of ileal bile acid transport for reducing cholesterololemia

(76) Inventor: Ann-Margret Lindqvist, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 10/502,355

(22) PCT Filed: Jan. 23, 2003
(86) PCT No.: PCT/GB03/00350

(30) Foreign Application Priority Data
Jan. 26, 2002 (GB) 0201850.5

(51) Int. Cl. A61K 31/7052; A61K 31/554; A61K 31/401; A61K 31/366; A61K 31/225

(52) U.S. Cl. 514/23; 514/211.09; 514/211.07; 514/548; 514/460; 514/423

(57) ABSTRACT

The use of an ileal bile acid transport (IBAT) inhibitor and the use of a combination of an IBAT inhibitor and an HMG CoA reductase inhibitor in the treatment of a warm-blooded animal, such as man, with hypercholesterolemia and/or other forms of dyslipidaemia wherein said hypercholesterolemia and dyslipidaemias are characterized by defects in lipoproteins or their receptors is described.

The present invention relates to compounds and combinations for the treatment of patients with hypercholesterolemia and/or other forms of dyslipidaemia wherein said hypercholesterolemia and dyslipidaemias are characterized by defects in lipoproteins or their receptors. These patients may manifest familial hypercholest erolemia, familial defective apolipoprotein B 100 or type III dyslipidaemia and these diseases may be of a heterozygous or homozygous nature. More specifically the invention relates to the use of an ileal bile acid transport (IBAT) inhibitor and the use of a combination of an IBAT inhibitor and an 3-hydroxy-3-methylglutaryl coenzyme A (HMG CoA) reductase inhibitor in the treatment of these diseases.

It is well-known that hyperlipidaemic conditions associated with elevated concentrations of total cholesterol and LDL cholesterol are major risk factors for cardiovascular atherosclerotic disease (Circulation 1999, 100, 1930-1938 and Circulation, 1999, 100, 1134-46). To reduce the risk and the total mortality due to cardiovascular disease, the reduction of plasma lipids, particularly LDL cholesterol, is now recognized as an important therapeutic goal (N Engl J. Med. 1995; 332:5, 12-21).

A large number of clinical trials have clearly established the HMG CoA reductase inhibitors—statins—as the primary drug of choice to accomplish this (Am J Cardiol. 1995; 76: 98C-106C; N Engl J. Med. 1998; 339: 1349-57; J Clin Epidemiol. 1992; 45: 849-60; Lancet. 1994; 344: 1383-9; Am J Cardiol 1998 Jul. 1; 82(I): 128]; Am J Cardiol. 1998; 81: 582-7) and in recent years novel, highly potent statins have emerged that can reduce plasma LDL cholesterol levels up to 60% (N Engl J. Med. 1999; 341: 70-6).

Interfering with the circulation of bile acids within the lumen of the intestinal tracts has also been found to reduce the level of cholesterol. Bile acids are synthesized in the liver from cholesterol and secreted into the bile. They are actively recycled (>95%) from the small intestine back to the liver. Previous established therapies have involved, for example, treatment with bile acid binders, such as resins. Frequently used bile acid binders are for instance cholestyramine and cholestipol. However, a study has found that resin treatment at high dose (2% cholestyramine) of LDL receptor deficient mice only marginally (<5%) reduces plasma cholesterol (Rudling & Angelin, Faseb J, 2001, 15, 1350-1356).

Another proposed therapy (Current Opinion on Lipidology, 1999, 10, 269-74) involves the treatment with substances with an IBAT inhibitory effect. Theoretically, IBAT inhibitors should have similar therapeutic effect as the resins but they might also be expected to have attractive advantages. First, it should be possible to administer IBAT inhibitors as tablets at the same dose intervals as statins. Second, they should not promote constipation, a laxative effect should instead be expected which may rather be a positive side effect, particularly in elderly patients. Third, a direct inhibition of the transport of bile acids across the ileum should be advantageous in situations when IBAT is upregulated. However available data on the effects of IBAT inhibitors is limited. Several IBAT agents have previously been shown to promote the fecal excretion of bile acids and to reduce plasma cholesterol. The proposed mechanism for the hypolipidaemic action of these compounds is by an increased number of hepatic LDL receptors due to the increased consumption of hepatic cholesterol caused by a compensatory increased bile acid synthesis (Arterioscler Thromb Vasc Biol. 1998; 18: 1304-11).

In the literature IBAT inhibitors are often referred to by different names. It is to be understood that where IBAT inhibitors are referred to herein, this term also encompasses compounds known in the literature as:
i) ileal apical sodium co-dependent bile acid transporter (ASBT) inhibitors;
ii) bile acid transporter (BAT) inhibitors;
iii) ileal sodium/bile acid cotransporter system inhibitors;
iv) apical sodium-bile acid cotransporter inhibitors;
v) ileal sodium-dependent bile acid transport inhibitors;
vi) bile acid reabsorption (BARI's) inhibitors; and
vii) sodium bile acid transporter (SBAT) inhibitors;
   where they act by inhibition of IBAT.

Familial hypercholesterolemia is due to an inherited autosomal dominant deficiency of LDL receptor expression on the cell surface, leading to excess concentrations of plasma total and LDL cholesterol followed by severe premature atherosclerosis. Familial hypercholesterolemia affects approximately 1 in 500 persons in the heterozygous state and approximately 1 in 1 million persons in the homozygous state. However, despite the efficiency of different statins (noted above), some patients with homozygous and heterozygous familial hyperlipoproteinemia may not achieve target LDL cholesterol levels when treated with these agents (even at the highest recommended dosage).

Familial defective apolipoprotein B-100 is a genetic disorder caused mainly by a substitution of glutamine for arginine at residue 3500 of the apolipoprotein B-100 molecule in the ligand that binds LDL to the LDL receptor. The result of this substitution is high levels of LDL because the abnormal LDL does not recognize the receptors and therefore the particles cannot be removed from circulation. In people of Western European descent, one person in 500 has a mutation in the Apo B-100 gene. The mutation that causes familial defective apolipoprotein B-100 is the most common mutation.

Patients with type III dyslipidemias often manifest different types of xanthomas as well as both hypercholesterolemia and hypertriglyceridemia. The underlying lipid disorder is characterized by abnormalities in VLDL and remnant IDL (Intermediate Density Lipoproteins) particles due to retarded clearance of the apoB containing particles. These patients also manifest abnormalities in the ApoE (isoforms, polymorphisms, mutations in E2/2, E3/3, E 4/4).

It is known that improvements in total and LDL cholesterol levels (and also the composition of lipids and apolipoproteins and their interrelations in other lipoproteins other than LDL) in patients with familial hypercholesterolemia can be made when statin therapy is combined with LDL apheresis (J Clin Basic Cardiol 2001; 4: 139). LDL apheresis is an aggressive blood transfusion technique where the patient's blood is separated into cells and plasma. The plasma is diverted over a column containing a material that locks onto the LDL cholesterol and removes it without removing the high density lipoprotein (HDL) cholesterol. The plasma is then returned to the patient. However the results are temporary, LDL apheresis is not a cure, and it needs to be repeated regularly.

There is clearly a need to improve the plasma LDL cholesterol levels with drug therapy in patients with hypercholesterolemia and/or other forms of dyslipidaemia wherein said hypercholesterolemia and dyslipidaemias are characterized by defects in lipoproteins or their receptors.

The present inventors have evaluated the effects of an IBAT inhibitor on plasma lipoproteins and hepatic cholesterol and bile acid metabolism in a situation where LDL receptors and ApoE are absent. In addition, in the same model, the effects of combining an IBAT inhibitor with a statin were also evaluated.

We have surprisingly found that in LDL receptor deficient and ApoE deficient double knock-out mice (LDL receptor/ApoE deficient), IBAT inhibition for only 3 days reduces plasma cholesterol dose dependently up to 40%. This finding of a strong reduction of plasma cholesterol in a situation where ApoE and LDL receptors are absent is surprising because it shows that (contrary to what was thought previously) the reduction of plasma cholesterol does not necessarily require hepatic LDL receptors or structures dependent on ApoE.

Furthermore, addition of a statin (in this case atorvastatin calcium salt) to IBAT inhibition further reduced plasma cholesterol by 24% so that the combined therapy resulted in a 64% reduction as compared to untreated animals.

In addition, in both studies, HDL cholesterol levels were increased. Thus, the IBAT inhibitor counteracted the HDL cholesterol lowering induced by atorvastatin calcium salt. The data suggests that when IBAT inhibitor is given in monotherapy the lipoprotein remnants (LP-remnants) and LDL cholesterol are reduced and HDL cholesterol is increased in a situation where LDL receptors and ApoE are absent. In combination therapy with statins the IBAT inhibitor acts synergistically in that the atherogenic ratio of LP-remnants and LDL cholesterol/HDL cholesterol is reduced by 71%.

Therefore according to the present invention, there is provided a method of treating hypercholesterolemia and/or other forms of dyslipidaemia wherein said hypercholesterolemia and dyslipidaemias are characterized by defects in lipoproteins or their receptors in a warm-blooded animal, such as man, in need of such treatment which comprises administering to said animal an effective amount of an IBAT inhibitor, or a pharmaceutically acceptable salt, solvate, solvate of such a salt or a prodrug thereof.

Therefore according to a further feature of the present invention, there is provided a method of treating hypercholesterolemia and/or other forms of dyslipidaemia wherein said hypercholesterolemia and dyslipidaemias are characterized by defects in lipoproteins or their receptors, in a warm-blooded animal, such as man, in need of such treatment which comprises administering to said animal an effective amount of an IBAT inhibitor, or a pharmaceutically acceptable salt, solvate, solvate of such a salt or a prodrug thereof in combination with an effective amount of an HMG Co-A reductase inhibitor, or a pharmaceutically acceptable salt, solvate, solvate of such a salt or a prodrug thereof.

Herein where the phrase “defects in lipoproteins or their receptors” is used this term means defects in LDL and/or the LDL receptor and/or ApoE and/or the ApoE receptor and/or the interaction and/or binding between these lipoproteins and their receptors. In one aspect of the invention this term means defects in LDL. In one aspect of the invention this term means defects in the LDL receptor. In one aspect of the invention this term means defects in ApoE. In one aspect of the invention this term means defects in the ApoE receptor. In one aspect of the invention this term means defects in the interactions between these lipoproteins and their receptors. In one aspect of the invention this term means defects in the binding between these lipoproteins and their receptors.

Herein, where the term “defects” is used in terms of lipoproteins or their receptors it is to be understood that this term means that the number of LDL receptors and/or ApoE receptors are less than adequate and may be totally deficient, and/or that the function of, and/or the response to physiological and/or pathological stimuli is inadequate resulting in hypercholesterolaemia and/or hypertriglicerideaemia.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the benefit of the synergistic effect of the combination.

In one aspect of the invention “hypercholesterolemia and/or other forms of dyslipidaemia wherein said hypercholesterolemia and dyslipidaemias are characterized by defects in lipoproteins or their receptors” is the disease state familial hypercholesterolemia.

In another aspect of the invention “hypercholesterolemia and/or other forms of dyslipidaemia wherein said hypercholesterolemia and dyslipidaemias are characterized by defects in lipoproteins or their receptors” is the disease state heterozygous familial hypercholesterolemia.

In a further aspect of the invention “hypercholesterolemia and/or other forms of dyslipidaemia wherein said hypercholesterolemia and dyslipidaemias are characterized by defects in lipoproteins or their receptors” is the disease state homozygous familial hypercholesterolemia.

In one aspect of the invention “hypercholesterolemia and/or other forms of dyslipidaemia wherein said hypercholesterolemia and dyslipidaemias are characterized by defects in lipoproteins or their receptors” is the disease state familial defective apolipoprotein B 100.

In another aspect of the invention “hypercholesterolemia and/or other forms of dyslipidaemia wherein said hypercholesterolemia and dyslipidaemias are characterized by defects in lipoproteins or their receptors” is the disease state heterozygous familial defective apolipoprotein B 100.

In a further aspect of the invention “hypercholesterolemia and/or other forms of dyslipidaemia wherein said hypercholesterolemia and dyslipidaemias are characterized by defects in lipoproteins or their receptors” is the disease state homozygous familial defective apolipoprotein B 100.

In one aspect of the invention “hypercholesterolemia and/or other forms of dyslipidaemia wherein said hypercholesterolemia and dyslipidaemias are characterized by defects in lipoproteins or their receptors” is the disease state type III dyslipidaemia.

In another aspect of the invention “hypercholesterolemia and/or other forms of dyslipidaemia wherein said hypercholesterolemia and dyslipidaemias are characterized by defects in lipoproteins or their receptors” is the disease state heterozygous type III dyslipidaemia.

In a further aspect of the invention “hypercholesterolemia and/or other forms of dyslipidaemia wherein said hypercholesterolermia and dyslipidaemias are characterized by defects in lipoproteins or their receptors” is the disease state homozygous type III dyslipidaemia.

Suitable compounds possessing IBAT inhibitory activity have been described, see for instance the compounds described in WO 93/16055, WO 94/18183, WO 94/18184, WO 96/05188, WO 96/08484, WO 96/16051, WO 97/33882, WO 98/38182, WO 99/35135, WO 98/40375, WO 99/35153, WO 99/64409, WO 99/64410, WO 00/01687, WO 00/47568, WO 00/61568, WO 01/68906, DE 19825804, WO 00/38725, WO 00/38726, WO 00/38727, WO 00/38728, WO 00/38729, WO 01/68906, WO 01/66533 and EP 864 582 and the contents of these patent applications, particularly the compounds described in claim 1 and the named examples, are incorporated herein by reference. Further suitable compounds include those described in WO94/24087, WO98/07749, WO 98/56757, WO 99/32478, WO 00/20437, WO 00/20392, WO 00/20393, WO 00/20410, WO 00/35889, WO 01/34570, WO 01/68637, WO 02/08211, WO 02/50051, JP 10072371, U.S. Pat. No. 5,070,103, EP 251 315, EP 417 725, EP 489 423, EP 549 967, EP 573 848, EP 624 593, EP 624 594, EP 624 595, EP 623 596, EP 869 121, and EP 1 070 703, and the contents of these patent applications, particularly the compounds described in claim 1 and the named examples, are incorporated herein by reference.

Particular classes of IBAT inhibitors suitable for use in the present invention are benzothiepines, and the compounds described in the claims of WO 00/01687, WO 96/08484 and WO 97/33882 are incorporated herein by reference. Other suitable classes of IBAT inhibitors are the 1,2-benzothiazepines, 1,4-benzothiazepines and 1,5-benzothiazepines. A further suitable class of IBAT inhibitors is the 1,2,5-benzothiadiazepines.

On particular suitable compound possessing IBAT inhibitory activity is (3R,5R)-3-butyl-3-ethyl-1,1-dioxido-5-phenyl-2,3,4,5-tetrahydro-1,4-benzothiazepin-8-yl β-D-glucopyranosiduronic acid (EP 864 582).

A further suitable compound possessing IBAT inhibitory activity is S-8921 (EP 597 107).

Additional suitable compounds possessing IBAT inhibitory activity have the following structure of formula (AI): 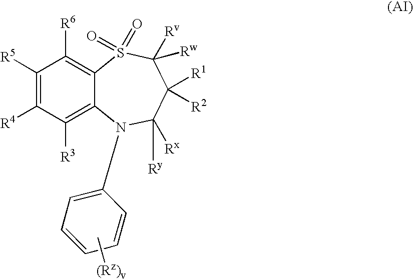
wherein:
R^{v} and R^{w} are independently selected from hydrogen or C₁₋₆alkyl;
R¹ and R² are independently selected from C₁₋₆alkyl;
R^{x} and R^{y} are independently selected from hydrogen or C₁₋₆alkyl, or one of R^{x} and R^{y} is hydrogen or C₆alkyl and the other is hydroxy or C₁₋₆alkoxy,
R^{z} is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, ureido, N′-(C₁₋₆alkyl)ureido, N-(C₁₋₆alkyl)ureido, N′,N′-(C₁₋₆alkyl)₂ureido, N′-(C₁₋₆alkyl)-N-(C₁₋₆alkyl)ureido, N′,N′-(C₁₋₆alkyl)₂—N-(C₁₋₆alkyl)ureido, N-(C₁₋₆alkyl)sulphamoyl and N,N-(C₁₋₆alkyl)₂sulphamoyl;
v is 0-5;
one of R⁴ and R⁵ is a group of formula (AIA): 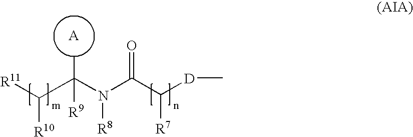
R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;
D is —O—, —N(R^{a})—, —S(O)_{b}— or —CH(R^{a})—; wherein R^{a} is hydrogen or C₁₋₆alkyl and b is 0-2;
Ring A is aryl or heteroaryl; wherein Ring A is optionally substituted by one or more substituents selected from R¹⁷;
R⁷ is hydrogen, C₁₋₄alkyl, carbocyclyl or heterocyclyl; wherein R⁷ is optionally substituted by one or more substituents selected from R¹⁸;
R⁸ is hydrogen or C₁₋₄alkyl;
R⁹ is hydrogen or C₁₋₄alkyl;
R¹⁰ is hydrogen, C₁₋₄alkyl, carbocyclyl or heterocyclyl; wherein R¹⁰ is optionally substituted by one or more substituents selected from R¹⁸;
R¹¹ is carboxy, sulpho, sulphino, phosphono, tetrazolyl, —P(O)(OR^{c})(OR^{d}), —P(O)(OH)(OR^{c}), —P(O)(OH)(R^{d}) or —P(O)(R^{d}) wherein R^{c} and R^{d} are independently selected from C₁₋₆alkyl; or R¹¹ is a group of formula (AIB): 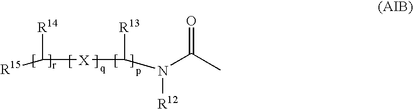
   wherein:
X is —N(R^{q})—, —N(R^{q})C(O)—, —O—, and —S(O)ₐ—; wherein a is 0-2 and R^{q} is hydrogen or
R¹² is hydrogen or C₁₋₄alkyl;
R¹³ and R¹⁴ are independently selected from hydrogen, C₁₋₄alkyl, carbocyclyl, heterocyclyl or R²³; wherein said C₁₋₄alkyl, carbocyclyl or heterocyclyl may be independently optionally substituted by one or more substituents selected from R²⁰;
R¹⁵ is carboxy, sulpho, sulphino, phosphono, tetrazolyl, —P(O)(OR^{e})(OR^{f}), —P(O)(OH)(OR^{e}), —P(O)(OH)Rₑ) or —P(O)(OR^{e})(R^{f}) wherein R^{e} and R^{f} are independently selected from C₁₋₆alkyl; or R¹⁵ is a group of formula (AIC): 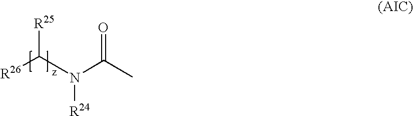
   wherein:
R²⁴ is selected from hydrogen or C₁₋₄alkyl;
R²⁵ is selected from hydrogen, C₁₋₄alkyl, carbocyclyl, heterocyclyl or R²⁷; wherein said C₁₋₄alkyl, carbocyclyl or heterocyclyl may be independently optionally substituted by one or more substituents selected from R²⁸;
R²⁶ is selected from carboxy, sulpho, sulphino, phosphono, tetrazolyl, —P(O)(OR^{g})(OR^{h}), —P(O)(OH)(OR^{g}), —P(O)(OH)R^{g}) or —P(O)(OR^{g})(R^{h}) wherein R^{g} and R^{h} are independently selected from C₁₋₆alkyl;
p is 1-3; wherein the values of R¹³ may be the same or different;
q is 0-1;
r is 0-3; wherein the values of R¹⁴ may be the same or different;
m is 0-2; wherein the values of R¹⁰ may be the same or different;
n is 1-3; wherein the values of R⁷ may be the same or different;
z is 0-3; wherein the values of R²⁵ may be the same or different;
R¹⁶, R¹⁷ and R¹⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alky)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R¹⁶, R¹⁷ and R¹⁸ may be independently optionally substituted on carbon by one or more R²¹;
R¹⁹, R²⁰, R²³, R²⁷ and R²⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹, R²⁰, R²³, R²⁷ and R²⁸ may be independently optionally substituted on carbon by one or more R²²;
R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, alkyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
   or a pharmaceutically acceptable salt, solvate, solvate of such a salt or a prodrug thereof.

Particular compounds of formula (AI) are:
1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(carboxymethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3-butyl-3-ethyl-5-phenyl-7-methylthio-8-(N-{R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-sulphoethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-sulphoethyl) carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(5-carboxypentyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{α-[N′-(2-sulphoethyl)carbamoyl]-2-fluorobenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-x-[N′-(R)-(2-hydroxy-1-carboxyethyl)carbamoyl]benzyl}carbamoylmethoxy)-2,3,4,5-tetrahydro-1,5-benzothuazepine;
1,1-dioxo-3,3-dibutyl-5-phenyl-7-methylthio-8-{N-[(R)-α-(N′-{(R)-1-[N″-(R)-(2-hydroxy-1-carboxyethyl)carbamoyl]-2-hydroxyethyl}carbamoyl)benzyl]carbamoylmethoxy}-2,3,4,5-tetrahydro-1,5-benzothiazepine;
1,1-dioxo-3-butyl-3-ethyl-5-phenyl-7-methylthio-8-(N-{α-[N′-(carboxymethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{α-[N′-((ethoxy)(methyl)phosphoryl-methyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-{N-[R)-x-(N′-{2-[(hydroxy)(methyl)phosphoryl]ethyl}carbamoyl)benzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[N′-(2-methylthio-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N-[(R)-α-(N′-{2-[(methyl)(ethyl) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-{N-[(R)-α-(N′-{2-[(methyl)(hydroxy) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[(R)-N′-(2-methylsulphinyl-1-carboxyethyl)carbamoyl]benzyl}carbamoylmethoxy)-2,3,4,5-tetrahydro-1,5-benzothiazepine; and
1,1-dioxo-3,3-dibutyl-5-phenyl-7-methoxy-8-[N-{(R)-α-[N′-(2-sulphoethyl)carbamoyl]-4-hydroxybenzyl}carbamoylmethoxy]-2,3,4,5-tetrahydro-1,5-benzothiazepine;
   or a pharmaceutically acceptable salt, solvate, solvate of such a salt or a prodrug thereof.

Further suitable compounds possessing IBAT inhibitory activity have the following 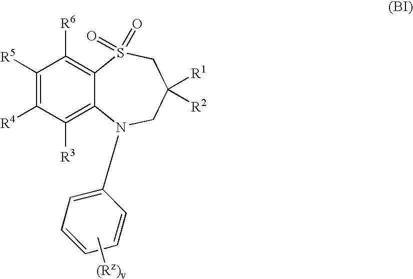
wherein:

One of R¹ and R² are selected from hydrogen or C₁₋₆alkyl and the other is selected from C₁₋₆alkyl;
R^{z} is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆-alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;
v is 0-5;
one of R⁴ and R⁵ is a group of formula (BIA): 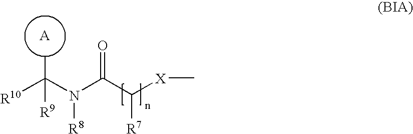
R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆-alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O), wherein a is 0 to 2, C₁₋₆alkoxycarbonyl, N-(C₁₋₆alkyl)sulphamoyl and N,N-(C₁₋₆alkyl)₂sulphamoyl; wherein R³ and R⁶ and the other of R⁴ and R⁵ may be optionally substituted on carbon by one or more R¹⁷;
X is —O—, —N(R^{a})—, —S(O)_{b}— or —CH(R^{a})—; wherein R^{a} is hydrogen or C₁₋₆alkyl and b is 0-2;
Ring A is aryl or heteroaryl; wherein Ring A is optionally substituted on carbon by one or more substituents selected from R¹⁸;
R⁷ is hydrogen, C₁₋₆alkyl, carbocyclyl or heterocyclyl; wherein R⁷ is optionally substituted on carbon by one or more substituents selected from R¹⁹; and wherein if said heterocyclyl contains an —NH— group, that nitrogen may be optionally substituted by a group selected from R²⁰;
R⁸ is hydrogen or C₁₋₆alkyl;
R⁹ is hydrogen or C₁₋₆alkyl;
R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkyenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃amino, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀-alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀aloyl, carbocyclyl-(C₁₋₁₀alkylene)ₚ—R²¹—(C₁₋₁₀alkylene)_{q}— or heterocyclyl-(C₁₋₁₀alkylene)ᵣ—R²²—(C₁₋₁₀alkylene)ₛ—; wherein R¹⁰ is optionally substituted on carbon by one or more substituents selected from R²³; and wherein if said heterocyclyl contains an —NH— group, that nitrogen may be optionally substituted by a group selected from R²⁴; or R¹⁰ is a group of formula (BIB): 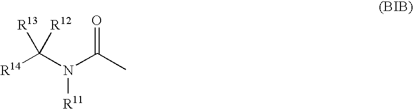
   wherein:
R¹ is hydrogen or C₁₋₆alkyl;
R¹² and R¹³ are independently selected from hydrogen, halo, nitro, cyano, hydroxy, amino, carbamoyl, mercapto, sulphamoyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an —NH—group, that nitrogen may be optionally substituted by a group selected from R²⁶;
R¹⁴ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, N,N-(C₁₋₁₀alkyl)₃ammonio, C₁₋₁₀ alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ—R²⁷—(C₁₋₁₀alkylene)_{q} or heterocyclyl-(C₁₋₁₀alkylene)ᵣ—R²⁸—(C₁₋₁₀alkylene)ₛ—; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (BIC):

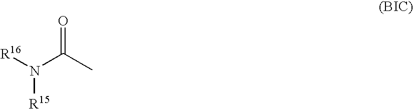

R¹⁵ is hydrogen or C₁₋₆alkyl;
R¹⁶ is hydrogen or C₁₋₆alkyl; wherein R¹⁶ may be optionally substituted on carbon by one or more groups selected from R³¹;
n is 1-3; wherein the values of R⁷ may be the same or different;
R¹⁷, R¹⁸, R¹⁹, R²³, R²⁵, R²⁹ or R³¹ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ—R³²—(C₁₋₁₀alkylene)_{q}— or heterocyclyl-(C₁₋₁₀alkylene)ᵣ—R³³—(C₁₋₁₀alkylene)ₛ—; wherein R¹⁷, R¹⁸, R¹⁹, R²³, R²⁵, R²⁹ or R³¹ may be independently optionally substituted on carbon by one or more R³⁴; and wherein if said heterocyclyl contains an —NH— group, that nitrogen may be optionally substituted by a group selected from R³⁵;
R²¹, R²², R²⁷, R²⁸, R³² or R³³ are independently selected from —O—, —NR³⁶—, —S(O)ₓ—, —NR³⁶C(O)NR³⁶—, —NR³⁶C(S)NR³⁶—, —OC(O)N═C—, —NR³⁶C(O)— or —C(O)NR³⁶—; wherein R³⁶ is selected from hydrogen or C₁₋₆alkyl, and x is 0-2;
p, q, r and s are independently selected from 0-2;
R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, alkyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;
R²⁰, R²⁴, R²⁶, R³⁰ or R³⁵ are independently selected from C₁₋₆alkyl, C₁₋₆alkanoyl, C₁₋₆alkylsulphonyl, C₁₋₆alkoxycarbonyl, carbamoyl, N-(C₁₋₆alkyl)carbamoyl, N,N-(C₁₋₆alkyl)carbamoyl, benzyl, benzyloxycarbonyl, benzoyl and phenylsulphonyl;
   or a pharmaceutically acceptable salt, solvate, solvate of such a salt or a prodrug thereof.

Further suitable compounds possessing IBAT inhibitory activity have the following structure of formula (CI): 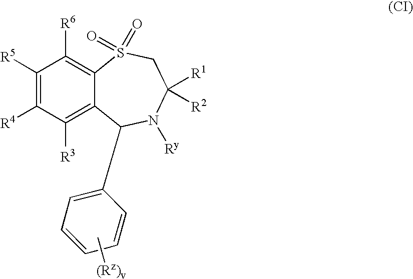
wherein:

One of R¹ and R² are selected from hydrogen or C₁₋₆alkyl and the other is selected from C₁₋₆alkyl;
R^{y} is selected from hydrogen, hydroxy, C₁₋₆alkyl, C₁₋₄alkoxy and C₁₋₆alkanoyloxy,
R^{x} is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C-₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;
v is 0-5;
one of R⁴ and R⁵ is a group of formula (CIA): 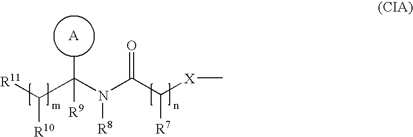
R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;
X is —O—, —N(R^{a})—, —S(O)_{b}— or —CH(R^{a})—; wherein R^{a} is hydrogen or C₁₋₆alkyl and b is 0-2;
Ring A is aryl or heteroaryl; wherein Ring A is optionally substituted by one or more substituents selected from R¹⁷;
R⁷ is hydrogen, C₁₋₄alkyl, carbocyclyl or heterocyclyl; wherein R⁷ is optionally substituted by one or more substituents selected from R¹⁸;
R⁸ is hydrogen or C₁₋₄alkyl;
R⁹ is hydrogen or C₁₋₄alkyl;
R¹⁰ is hydrogen, C₁₋₄alkyl, carbocyclyl or heterocyclyl; wherein R¹⁰ is optionally substituted by one or more substituents selected from R¹⁹;
R¹¹ is carboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein R^{e} and R^{d} are independently selected from C₁₋₆alkyl; or R¹¹ is a group of formula (CIB): 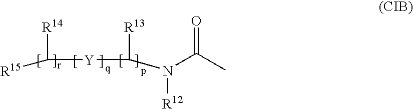
   wherein:
Y is —N(R^{x})—, —N(R^{x})C(O)—, —O—, and —S(O)ₐ—; wherein a is 0-2 and R^{x} is hydrogen or C₁₋₄alkyl;
R¹² is hydrogen or C₁₋₄alkyl;
R¹³ and R¹⁴ are independently selected from hydrogen, C₁₋₄alkyl, carbocyclyl or heterocyclyl; wherein R¹³ and R¹⁴ may be independently optionally substituted by one or more substituents selected from R²⁰;
R¹⁵ is carboxy, sulpho, sulphino, phosphono, —P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from C₁₋₆alkyl;
p is 1-3; wherein the values of R¹³ may be the same or different;
q is 0-1;
r is 0-3; wherein the values of R¹⁴ may be the same or different;
m is 0-2; wherein the values of R¹⁰ may be the same or different;
n is 1-3; wherein the values of R⁷ may be the same or different;
R¹⁶, R¹⁷ and R¹⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄oxycarbonyl, N-(C₁₋₄alkyl)sulphamoyl and N,N-(C₁₋₄alkyl)₂sulphamoyl; wherein R¹⁶, R¹⁷ and R¹⁸ may be independently optionally substituted on carbon by one or more R²¹;
R¹⁹ and R²⁰ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐwherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹ and R²⁰ may be independently optionally substituted on carbon by one or more R
R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, alkyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
   or a pharmaceutically acceptable salt, solvate, solvate of such a salt or a prodrug thereof.

Further suitable compounds possessing BAT inhibitory activity have the following structure of formula (DI): 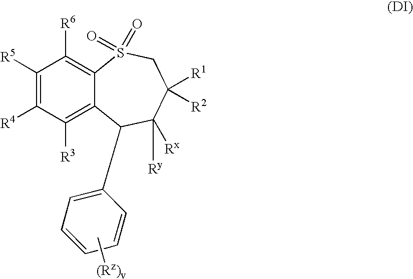
wherein:

One of R¹ and R² are selected from hydrogen or C₁₋₆alkyl and the other is selected from C₁₋₆alkyl;
R^{x} and R^{y} are independently selected from hydrogen, hydroxy, amino, mercapto, C₁₋₆alkyl, C₁₋₆alkoxy, N-(C₁₋₆alkyl)amino, N,N-(C₁₋₆alkyl)₂amino, C₁₋₆alkylS(O)ₐ wherein a is 0 to 2;
R^{z} is selected from halo, nitro, cyano, hydroxy, amino, carboxy, carbamoyl, mercapto, sulphamoyl, C₁₋₆alkyl, C₂₋₆alkenyl, C₂₋₆alkyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;
v is 0-5;
one of R⁴ and R⁵ is a group of formula (DIA): 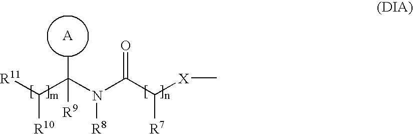
R³ and R⁶ and the other of R⁴ and R¹ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;
X is —O—, —N(R^{a})—, —S(O)_{b}— or —CH(R^{a})—; wherein R^{a} is hydrogen or C₁₋₆alkyl and b is 0-2;
Ring A is aryl or heteroaryl; wherein Ring A is optionally substituted by one or more substituents selected from R¹⁷;
R⁷ is hydrogen, C₁₋₄alkyl, carbocyclyl or heterocyclyl; wherein R⁷ is optionally substituted by one or more substituents selected from R¹⁸;
R⁸ is hydrogen or C₁₋₄alkyl;
R⁹ is hydrogen or C₁₋₄alkyl;
R¹⁰ is hydrogen, C₁₋₄alkyl, carbocyclyl or heterocyclyl; wherein R¹⁰ is optionally substituted by one or more substituents selected from R¹⁹;
R¹¹ is carboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆alkyl; or R¹¹ is a group of formula (DIB): 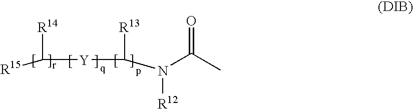
   wherein:
Y is —N(Rⁿ)—, —N(Rⁿ)C(O)—, —O—, and —S(O)a-; wherein a is 0-2 and Rⁿ is hydrogen or C₁₋₄alkyl;
R¹² is hydrogen or C₁₋₄alkyl;
R¹³ and R¹⁴ are independently selected from hydrogen, C₁₋₄alkyl, carbocyclyl or heterocyclyl; wherein R¹³ and R¹⁴ may be independently optionally substituted by one or more substituents selected from R²⁰;
R¹⁵ is carboxy, sulpho, sulphino, phosphono, —P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from C₁₋₆alkyl;
p is 1-3; wherein the values of R¹³ may be the same or different;
q is 0-1;
r is 0-3; wherein the values of R¹⁴ may be the same or different;
m is 0-2; wherein the values of R¹⁰ may be the same or different;
n is 1-3; wherein the values of R⁷ may be the same or different;
R¹⁶, R¹⁷ and R¹⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R¹⁶, R¹⁷ and R¹⁸ may be independently optionally substituted on carbon by one or more R²¹;
R¹⁹ and R²⁰ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹ and R²⁰ may be independently optionally substituted on carbon by one or more R²²;
R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, alkyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
   or a pharmaceutically acceptable salt, solvate, solvate of such a salt or a prodrug thereof.

Further suitable compounds possessing IBAT inhibitory activity have the following structure of formula (EI): 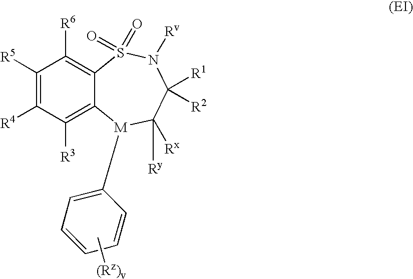
wherein:

R^{v} is selected from hydrogen or C₁₋₆alkyl;
One of R¹ and R² are selected from hydrogen or C₁₋₆alkyl and the other is selected from C₁₋₆alkyl;
R^{x} and R^{y} are independently selected from hydrogen, hydroxy, amino, mercapto, C₁₋₆alkyl, C₁₋₆alkoxy, N-(C₁₋₆alkyl)amino, N,N-(C₁₋₆alkyl)₂amino, C₁₋₆alkylS(O)ₐ wherein a is 0 to 2;
M is selected from —N— or —CH—;
R^{x} is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;
v is 0-5;
one of R⁴ and R⁵ is a group of formula (EIA): 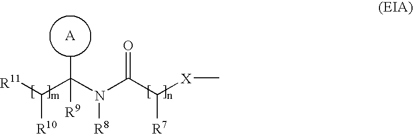
R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;
X is —O—, —N(R^{a})—, —S(O)_{b}— or —CH(R^{a})—; wherein R^{a} is hydrogen or C₁₋₆alkyl and b is 0-2;
Ring A is aryl or heteroaryl; wherein Ring A is optionally substituted by one or more substituents selected from R¹⁷;
R⁷ is hydrogen, C₁₋₄alkyl, carbocyclyl or heterocyclyl; wherein R⁷ is optionally substituted by one or more substituents selected from R¹⁸;
R⁸ is hydrogen or C₁₋₄alkyl;
R⁹ is hydrogen or C₁₋₄alkyl;
R¹⁰ is hydrogen, C₁₋₄alkyl, carbocyclyl or heterocyclyl; wherein R¹⁰ is optionally substituted by one or more substituents selected from R¹⁹;
R¹¹ is caboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆alkyl; or R¹¹ is a group of formula (EIB): 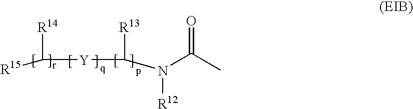
   wherein:

Y is —N(Rⁿ)—, —N(Rⁿ)C(O)—, —O—, and —S(O)a-; wherein a is 0-2 and Rⁿ is hydrogen or C₁₋₄alkyl;
R¹² is hydrogen or C₁₋₄alkyl;
R¹³ and R¹⁴ are independently selected from hydrogen, C₁₋₄alkyl, carbocyclyl or heterocyclyl; wherein R¹³ and R¹⁴ may be independently optionally substituted by one or more substituents selected from R²⁰;
R¹⁵ is carboxy, sulpho, sulphino, phosphono, —P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from C₁₋₆alkyl;
p is 1-3; wherein the values of R¹³ may be the same or different;
q is 0-1;
r is 0-3; wherein the values of R¹⁴ may be the same or different;
m is 0-2; wherein the values of R¹⁰ may be the same or different;
n is 1-3; wherein the values of R⁷ may be the same or different;
R¹⁶, R¹⁷ and R¹⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐwherein a is 0 to 2, C₁₋₄alkoxycarbonyl, N-(C₁₋₄alkyl)sulphamoyl and N,N-(C₁₋₄alkyl)₂sulphamoyl; wherein R¹⁶, R¹⁷ and R¹⁸ may be independently optionally substituted on carbon by one or more R²¹;
R¹⁹ and R²⁰ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ, wherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹ and R²⁰ may be independently optionally substituted on carbon by one or more R²²;
R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, alkyl, ethynyl, methoxycarbonyl, formyl acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
   or a pharmaceutically acceptable salt, solvate, solvate of such a salt or a prodrug thereof.

Further suitable compounds possessing IBAT inhibitory activity have the following structure of formula (FI): 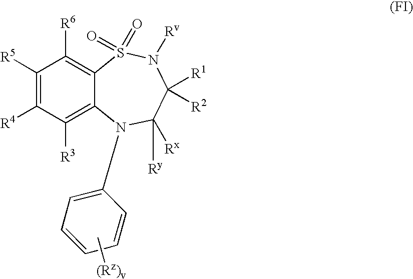
wherein:
R^{v} is selected from hydrogen or C₁₋₆alkyl;
One of R¹ and R² are selected from hydrogen or C₁₋₆alkyl and the other is selected from C₁₋₆alkyl;
R^{x} and R^{y} are independently selected from hydrogen, hydroxy, amino, mercapto, C₁₋₆alkyl, C₁₋₆alkoxy, N-(C₁₋₆alkyl)amino, N,N-(C₁₋₆alkyl)₂amino, C₁₋₆alkylS(O)ₐ wherein a is 0 to 2;
R^{z} is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;
v is 0-5;
one of R⁴ and R⁵ is a group of formula (FIA): 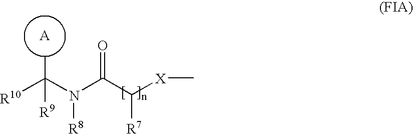
R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl; wherein R³ and R⁶ and the other of R¹ and R⁵ may be optionally substituted on carbon by one or more R¹⁷;
X is —O—, —N(R^{a})—, —S(O)_{b}— or —CH(R^{a})—; wherein R^{a} is hydrogen or C₁₋₆alkyl and b is 0-2;
Ring A is aryl or heteroaryl; wherein Ring A is optionally substituted on carbon by one or more substituents selected from R¹⁸;
R⁷ is hydrogen, C₁₋₆alkyl, carbocyclyl or heterocyclyl; wherein R⁷ is optionally substituted on carbon by one or more substituents selected from R¹⁹; and wherein if said heterocyclyl contains an —NH— group, that nitrogen may be optionally substituted by a group selected from R²⁰;
R⁸ is hydrogen or C₁₋₆alkyl;
R⁹ is hydrogen or alkyl;
R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ—R²¹—(C₁₋₁₀alkylene)_{q}— or heterocyclyl-(C₁₋₁₀alkylene)ᵣ—R²²—(C₁₋₁₀alkylene)ₛ—; wherein R¹⁰ is optionally substituted on carbon by one or more substituents selected from R²³; and wherein if said heterocyclyl contains an —NH— group, that nitrogen may be optionally substituted by a group selected from R²⁴; or R¹⁰ is a group of formula (FIB): 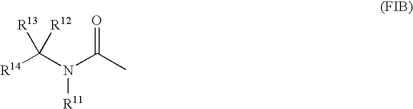
   wherein:
R¹¹ is hydrogen or C₁₋₆alkyl;
R¹² and R¹³ are independently selected from hydrogen, halo, carbamoyl, sulphamoyl, C₁₋₁₀alkyl, C₂₋₁₀alkenyl, C₂₋₁₀alkynyl, C₁₋₁₀alkanoyl, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an —NH— group, that nitrogen may be optionally substituted by a group selected from R²⁶;
R¹⁴ is selected from hydrogen, halo, carbamoyl, sulphamoyl, hydroxyaminocarbonyl, C₁₋₁₀alkyl, C₂₋₁₀alkenyl, C₂₋₁₀alkynyl, C₁₋₁₀alkanoyl, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₚ—R²⁷—(C₁₋₁₀alkylene)_{q}— or heterocyclyl-(C₁₋₁₀alkylene), —R²⁸—(C₁₋₁₀alkylene)ₛ—; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (IC): 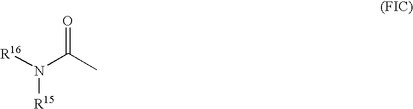
R¹⁵ is hydrogen or C₁₋₆alkyl;
R¹⁶ is hydrogen or C₁₋₆alkyl; wherein R¹⁶ may be optionally substituted on carbon by one or more groups selected from R³¹;
n is 1-3; wherein the values of R⁷ may be the same or different;
R¹⁷, R¹⁸, R¹⁹, R¹, R²⁵, R²⁹ or R³¹ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino; N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₙ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ—R^{32—-(C}₁₋₁₀alkylene)_{q}— or heterocyclyl-(C₁₋₁₀alkylene)ᵣ—R³³—(C₁₋₁₀alkylene)ₛ—; wherein R¹⁷, R¹⁸, R¹⁹, R²³, R²⁵, R²⁹ or R³¹ may be independently optionally substituted on carbon by one or more R³⁴; and wherein if said heterocyclyl contains an —NH— group, that nitrogen may be optionally substituted by a group selected from R³⁵;
R²¹, R²², R²⁷, R²⁸, R³² or R³³ are independently selected from —O—, —NR³⁶—, —S(O)ₓ—, —NR³⁶C(O)NR³⁶—, —NR³⁶C(S)NR³⁶—, —OC(O)N═C—, —NR³⁶C(O)— or —C(O)NR³⁶—; wherein R³⁶ is selected from hydrogen or C₁₋₆alkyl, and x is 0-2;
p, q, r and s are independently selected from 0-2;
R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, alkyl, ethynyl, formyl, acetyl, formamido, acetylamino, acetoxy, methylamino, dimethylamino, N-methylcarbamoyl, N. N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;
R²⁰, R²⁴, R²⁶, R³⁰ or R³⁵ are independently selected from C₁₋₆alkyl, C₁alkanoyl, C₁₋₆alkylsulphonyl, C₁₋₆alkoxycarbonyl, carbamoyl, N-(C₁ alkyl)carbamoyl, N,N-(C₁₋₆alkyl)carbamoyl, benzyl, benzyloxycarbonyl, benzoyl and phenylsulphonyl; or a pharmaceutically acceptable salt, solvate, solvate of such a salt or a prodrug thereof.

Additional suitable IBAT inhibitors include any one of the following compounds:
1,1-dioxo-3,3-dibutyl-5-phenyl-7-methylthio-8-(N-{(R)-α-[N—((R)-1-carboxy-2-methylthio-ethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxy-2-(R)-hydroxypropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxybutyl) carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxypropyl) carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxyethyl) carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxy-2-(R)-hydroxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxyethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((R)-1-carboxy-2-methylthioethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-{(S)-1-[N—((S)-2-hydroxy-1-carboxyethyl)carbamoyl]propyl}carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N—((S)-1-carboxy-2-methylpropyl)carbamoyl]benzyl}carbamoylmethoxy)-2,3,4,5-tetrahydro-1,2,5-benzothiadiazepine;
1,1-Dioxo-3,3-dibutyl-5-phenyl-7-methylthio-8-(N-{(R)-α-[N—((S)-1-carboxypropyl) carbamoyl]-4-hydroxybenzyl}carbamoylmethoxy)-2,3,4,5-tetrahydro-1,2,5-benzothiadiazepine;
1,1-Dioxo 3,3-dibutyl-5-phenyl-7-methylthio-8-[N—((R/S)-α-{N-[1-(R)-2-(S)-1-hydroxy-1-(3,4-dihydroxyphenyl)prop-2-yl]carbamoyl}-4-hydroxybenzyl)carbamoylmethoxy]-2,3,4,5-tetrahydro-1,2,5-benzothiadiazepine;
1,1-Dioxo-3,3-dibutyl-5-phenyl-7-methylthio-8-(N-{(R)-x-[N-(2-(S)-3-(R)-4-(R)-5-R)-2,3,4,5,6-pentahydroxyhexyl)carbamoyl]-4-hydroxybenzyl}carbamoylmethoxy)-2,3,4,5-tetrahydro-1,2,5-benzothiadiazepine; and
1,1-Dioxo-3,3-dibutyl-5-phenyl-7-methylthio-8-(N-{(R)-α-[N-(2-(S)-3-(R)-4-(R)-5-(R)-2,3,4,5,6-pentahydroxyhexyl)carbamoyl]benzyl}carbamoylmethoxy)-2,3,4,5-tetrahydro-1,2,5-benzothiadiazepine;

Compounds of formula (AI), (BI), (CI), (DI), (EI) and (FI) or a pharmaceutically acceptable salt, solvate, solvate of such a salt or a prodrug thereof may be prepared by processes known in the art.

Suitable HMG Co-A reductase inhibitors, pharmaceutically acceptable salts, solvates, solvates of such salts or prodrugs thereof are statins well known in the art. Particular statins are fluvastatin, lovastatin, pravastatin, simvastatin, atorvastatin, cerivastatin, bervastatin, dalvastatin, mevastatin and rosuvastatin, or a pharmaceutically acceptable salt, solvate, solvate of such a salt or a prodrug thereof. A particular statin is atorvastatin, or a pharmaceutically acceptable salt, solvate, solvate of such a salt or a prodrug thereof A more particular statin is atorvastatin calcium salt. A further particular statin is rosuvastatin, or a pharmaceutically acceptable salt, solvate, solvate of such a salt or a prodrug thereof. A preferable particular statin is rosuvastatin calcium salt.

In a particular aspect of the invention an IBAT inhibitor or a pharmaceutically acceptable salt, solvate, solvate of such a salt or a prodrug thereof is an IBAT inhibitor or a pharmaceutically acceptable salt thereof.

In a further particular aspect of the invention an HMG CoA reductase inhibitor or a pharmaceutically acceptable salt, solvate, solvate of such a salt or a prodrug thereof is an HMG CoA reductase inhibitor or a pharmaceutically acceptable salt thereof.

Suitable pharmaceutically acceptable salts of the above compounds are, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric, acetate or maleic acid. In addition a suitable pharmaceutically acceptable salt of a compound which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds may be administered in the form of a pro-drug which is broken down in the human or animal body to give the parent compound. Examples of pro-drugs include in vivo hydrolysable esters and in vivo hydrolysable amides.

An in vivo hydrolysable ester of a compound containing carboxy or hydroxy group is, for example, a pharmaceutically acceptable ester which is hydrolysed in the human or animal body to produce the parent acid or alcohol. Suitable pharmaceutically acceptable esters for carboxy include C₁-alkoxymethyl esters for example methoxymethyl, C₁₋₆alkanoyloxymethyl esters for example pivaloyloxymethyl, phthalidyl esters, C₃₋₈-cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds.

An in vivo hydrolysable ester of a compound containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

A suitable value for an in vivo hydrolysable amide of a compound containing a carboxy group is, for example, a N—C₁₋₆alkyl or N,N-di-C₁₋₆alkyl amide such as N-methyl, N-ethyl, N-propyl, N,N-dimethyl, N-ethyl-N-methyl or N,N-diethyl amide.

Experimental

Material

In the following section Compound (I) refers to (3R,5R)-3-butyl-3-ethyl-1,1-dioxido-5-phenyl-2,3,4,5-tetrahydro-1,4-benzothiazepin-8-yl β-D-glucopyranosiduronic acid (EP 864582):

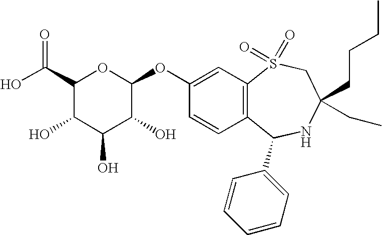

Atorvastatin calcium salt (40 mg tablets) was ground into fine particles and mixed into regular mouse R³-chow which then was pelleted (0.05% w:w). Compound (I) was dissolved in polyethanylglycol (PEG):ethanol:solutol:Water (4:1:0.5:8.5) vehicle and administered by gavage once a day in the afternoon.

Animals

Altogether 54 female LDL receptor/ApoE deficient mice were used (5 to 6 weeks old weighing 25 to 30 g at the start of the study; obtained from B&M/AS, Denmark). They were kept under standardized conditions with free access to water and chow. The light-cycle hours were between 6:00 a.m. and 6:00 p.m. In experiment I, the dose response study, the mice were treated with Compound (I) by gavage once a day in the afternoon the first three days and in the morning the last day. The control group on regular R³-chow received the vehicle by gavage. In experiment II, the combination study, atorvastatin calcium salt (0.05%) was mixed with R³ chow. The mice received atorvastatin calcium salt (0.05% in chow) and/or Compound (I) by gavage for 7 days. The control group received R³ chow and vehicle.

Plasma Collection

Mice were starved 3 hours before they were scarified at 10 a.m. Animals were anaesthetized with isofluran, bled by cardiac puncture, and thereafter killed by cervical dislocation. Blood was collected into EDTA containing tubes, plasma was separated by centrifugation and stored at −70° C.

Cholesterol Assay

Cholesterol in plasma and in FPLC on line measurement was performed with a commercial cholesterol kit from Roche Diagnostics, GmbH, Germany, Cholesterol, CHOD-PAP 1489437.

Triglyceride Assay

Triglycerides in plasma was measured by using a commercial reagent kit, from Roche Diagnostics, GmbH, Germany, Triglycerides/GB, 450032.

Size-Fractionation of Lipoproteins by Miniaturized On-Line FPLC.

The cholesterol distribution profiles were measured by using a size exclusion high performance liquid chromatography system, SMART, with column Superose 6 PC 3.2/30, (Amersham Pharmacia Biotec, Uppsala, Sweden). The chromatographic system was linked to an air segmented continuous flow system for online post-derivatization analysis of total cholesterol by using enzymatic colorimetric reagents. The SMART-system was connected to a sample injector, (Gina 50, Gynkotek HPLC, Germering, GmbH). Elution buffer consisted of 0.01M Tris, 0.03 M NaCl, pH 7.40, flow rate 35 ml/min. The on line flow system was equipped with a peristaltic pump, flow rate 0.7 ml/min, and an incubation coil for 8 minutes at 37° C. The absorbance was measured at 500=1 with a UV/VIS detector, (Jasco UV-970, Jasco International Co, Ltd, Japan). Data were integrated with a Chromeleon chromatography data system (Gynkotek HPLC, Germering GmbH). The distribution of lipoproteins was continuously measured as total cholesterol by using enzymatic calorimetric reagent, reconstituted in water, double volume compared to manufacturer instructions. The commercial kits were from Roche Diagnostics, GmbH, Germany, Cholesterol, CHOD-PAP 1489437. The separation was performed within 60 minutes on a 10 μl sample. The integrated area of the fractions was expressed in molar concentration. The various peaks in the profiles are designated LP-remnants, LDL, and HDL for simplicity, even though it is clear that the separation is determined primarily by the size of the lipoproteins.

Results

With the aim to determine the effect of the BAT inhibitor, Compound (I), on the level of plasma lipids in LDL receptor/ApoE deficient mice, groups of animals received vehicle or Compound (I) at increasing doses (0.62, 2.5, 10 and 40 μmol/kg/day) for 3.5 days. Compound (I) treatment reduced total plasma cholesterol dose dependently, and a 40% reduction was obtained at the highest dose. Plasma triglycerides were not significantly altered although a tendency for an increase was seen. To analyze plasma lipoproteins in detail, plasma lipoprotein patterns were generated after separation of plasma by FPLC. The results showed that the reduction in plasma cholesterol occurred in LP-remnants (63% reduction) and LDL (23% reduction) fractions, whereas there was no reduction of HDL cholesterol, if anything an increase was seen (Table 1).

TABLE 1
Treatment for three days of LDLreceptor/ApoE deficient receptor
knock-out mice with increasing doses of Compound (I)

| | | 0.625 | | | |
|---|---|---|---|---|---|
| | | μmol/ | 2.5 μmol/ | 10 μmol/ | 40 μmol/ |
| | Controls | kg/day | kg/day | kg/day | kg/day |
| Plasma lipids/ | 100% | % Change compared to control group | | | |
| lipoproteins | | | | | |
| TG¹ (mM) | 2.7 | 13 | 16 | 23 | 8 |
| Chol² (mM) | 20.1 | −9 | −28 | −35 | −40 |
| LP-remnants | 10.1 | −27 | −45 | −54 | −63 |
| (mM) | | | | | |
| LDL (mM) | 9.2 | 8 | −14 | −20 | −23 |
| HDL (mM) | 0.8 | 29 | 27 | 36 | 45 |
| LP-remnants + | 24.0 | −30 | −45 | −55 | −62 |
| LDL/HDL | | | | | |

¹TG = Plasma triglycerides
²Chol = plasma total cholesterol

Atorvastatin calcium salt alone (0.05% in diet approximately 80-100 mg/kg/day) reduced total plasma cholesterol by 25% whereas Compound (I) (10 μmol/kg/day) resulted in a 40% reduction. The combined treatment using both drugs resulted in a further reduction so that a 63% reduction was obtained (Table 2, FIG. 2). Atorvastatin calcium salt alone and the combination of atorvastatin calcium salt and Compound (I) reduced plasma triglycerides by 60% and 40% respectively. Compound (I) treatment alone had no effect on the plasma triglyceride level in this study. FPLC analysis of plasma (FIG. 1) showed that the reduction of cholesterol by atorvastatin calcium salt was limited to LDL particles only (44% reduction) while Compound (I) treatment strongly reduced both LDL (30% reduction) and LP-remnants cholesterol (62% reduction) (FIG. 2). The combination of the two drugs resulted in a further reduction of cholesterol particularly within LDL particles so that a 64% reduction was obtained. An increase (22%) in HDL cholesterol was seen after treatment with Compound (I) alone, or in combination with atorvastatin calcium salt (15%).

[image] TABLE 2
Plasma lipid levels in LDLreceptor/ApoE deficient double knock-out
mice after treatment with Compound (I), atorvastatin calcium salt
or combination of the two compounds for one week.

| | | | Atorvastatin | Combi- |
|---|---|---|---|---|
| | Controls | Compound(I) | calcium salt | nation |
| Plasma lipids/ | 100% | % Change compared to control group | | |
| lipoproteins | | | | |
| TG¹ (mM) | 2.1 | 7 | −59 | −43 |
| Chol² (mM) | 16.9 | −42 | −24 | −64 |
| LP-remnants (mM) | 7.6 | −62 | −2 | −69 |
| LDL (mM) | 8.7 | −29 | −43 | −64 |
| HDL (mM) | 0.6 | 22 | −22 | 15 |
| LP-remnants + | 25.8 | −54 | 2 | −71 |
| LDL/HDL | | | | |

¹TG = Plasma triglycerides
²Chol = Plasma total cholesterol

[image]

The combination of atorvastatin calcium salt and Compound (I) showed a synergistic effect on the ratio of (LP-remnants+LDL-cholesterol)/(HDL-cholesterol).

Therefore according to another aspect of the invention, there is provided a method of testing whether an IBAT inhibitor, or a pharmaceutically acceptable salt, solvate, solvate of such a salt or a prodrug thereof has any one of the following effects:
i) lowering total cholesterol; optionally in combination with an HMG CoA reductase inhibitor, or a pharmaceutically acceptable salt, solvate, solvate of such a salt or a prodrug thereof;
ii) lowering LP-remnants; optionally in combination with an HMG CoA reductase inhibitor, or a pharmaceutically acceptable salt, solvate, solvate of such a salt or a prodrug thereof;
iii) lowering LDL; optionally in combination with an HMG CoA reductase inhibitor, or a pharmaceutically acceptable salt, solvate, solvate of such a salt or a prodrug thereof;
iv) raising HDL; optionally in combination with an BIG CoA reductase inhibitor, or a pharmaceutically acceptable salt, solvate, solvate of such a salt or a prodrug thereof; or
v) exhibits a synergistic effect in combination with an BMG CoA reductase inhibitor, or a pharmaceutically acceptable salt, solvate; solvate of such a salt or a prodrug thereof on the lowering of the ratio of (LP-remnants+LDL-cholesterol)/(HDL-cholesterol); wherein the method of testing comprises administering the IBAT inhibitor, or a pharmaceutically acceptable salt, solvate, solvate of such a salt or a prodrug thereof, to a transgenic LDL receptor and/or ApoE deficient non-human mammal optionally in combination with an HMG CoA reductase inhibitor, or a pharmaceutically acceptable salt, solvate, solvate of such a salt or a prodrug thereof; and determining whether there has been an effect on any one of (i)-(v) above on the non human mammal.

In one aspect of the invention the non-human mammal is a rodent.

In another aspect of the invention the non-human mammal is a mouse.

In one aspect of the invention the method of testing relates to an BAT inhibitor, or a pharmaceutically acceptable salt, solvate, solvate of such a salt or a prodrug thereof without the HMG CoA reductase inhibitor, or a pharmaceutically acceptable salt, solvate, solvate of such a salt or a prodrug thereof.

In another aspect of the invention the method of testing relates to an IBAT inhibitor, or a pharmaceutically acceptable salt, solvate, solvate of such a salt or a prodrug thereof in combination with an HMG CoA reductase inhibitor, or a pharmaceutically acceptable salt, solvate, solvate of such a salt or a prodrug thereof.

In one aspect of the invention the method of testing relates to testing whether an IBAT inhibitor, or a pharmaceutically acceptable salt, solvate, solvate of such a salt or a prodrug thereof exhibits a synergistic effect in combination with an HMG CoA reductase inhibitor, or a pharmaceutically acceptable salt, solvate, solvate of such a salt or a prodrug thereof on the lowering of the ratio of (LP-remnants+LDL-cholesterol)/(HDL-cholesterol) In one aspect of the invention the transgenic non-human mammal is both LDL receptor and ApoE deficient.

Therefore according to the present invention, there is provided a method of treating hypercholesterolemia and/or other forms of dyslipidaemia wherein said hypercholesterolemia and dyslipidaemias are characterized by defects in lipoproteins or their receptors, in a warm-blooded animal, such as man, in need of such treatment which comprises administering to said animal an effective amount of an IBAT inhibitor, or a pharmaceutically acceptable salt, solvate, solvate of such a salt or a prodrug thereof.

Therefore according to the present invention, there is provided a method of lowering abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors, in a warm-blooded animal, such as man, in need of such treatment which comprises administering to said animal an effective amount of an IBAT inhibitor, or a pharmaceutically acceptable salt, solvate, solvate of such a salt or a prodrug thereof.

Therefore according to the present invention, there is provided a method of treating hypercholesterolemia and/or other forms of dyslipidaemia wherein said hypercholesterolemia and dyslipidaemias are characterized by defects in lipoproteins or their receptors, in a warm-blooded animal, such as man, in need of such treatment which comprises administering to said animal an effective amount of an IBAT inhibitor, or a pharmaceutically acceptable salt, solvate, solvate of such a salt or a prodrug thereof in combination with an effective amount of an HMG Co-A reductase inhibitor, or a pharmaceutically acceptable salt, solvate, solvate of such a salt or a prodrug thereof.

Therefore according to the present invention, there is provided a method of lowering abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors, in a warm-blooded animal, such as man, in need of such treatment which comprises administering to said animal an effective amount of an IBAT inhibitor, or a pharmaceutically acceptable salt, solvate, solvate of such a salt or a prodrug thereof in combination with an effective amount of an HMG Co-A reductase inhibitor, or a pharmaceutically acceptable salt, solvate, solvate of such a salt or a prodrug thereof.

According to a further aspect of the invention there is provided a pharmaceutical composition which comprises an IBAT inhibitor, or a pharmaceutically acceptable salt, solvate, solvate of such a salt or a prodrug thereof, in association with a pharmaceutically acceptable diluent or carrier for use in the treatment of hypercholesterolemia and/or other forms of dyslipidaemia wherein said hypercholesterolemia and dyslipidaemias are characterized by defects in lipoproteins or their receptors.

According to a further aspect of the invention there is provided a pharmaceutical composition which comprises an IBAT inhibitor, or a pharmaceutically acceptable salt, solvate, solvate of such a salt or a prodrug thereof, in association with a pharmaceutically acceptable diluent or carrier for use in lowering of abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

According to a further aspect of the invention there is provided a pharmaceutical composition which comprises an IBAT inhibitor, or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, in association with a pharmaceutically acceptable diluent or carrier for use in the treatment of hypercholesterolemia and/or other forms of dyslipidaemia wherein said hypercholesterolemia and dyslipidaemias are characterized by defects in lipoproteins or their receptors.

According to a further aspect of the invention there is provided a pharmaceutical composition which comprises an IBAT inhibitor, or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, in association with a pharmaceutically acceptable diluent or carrier for use in lowering of abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

According to a further aspect of the invention there is provided a pharmaceutical composition which comprises an IBAT inhibitor, or a pharmaceutically acceptable salt, solvate, solvate of such a salt or a prodrug thereof, in association with a pharmaceutically acceptable diluent or carrier, in combination with a pharmaceutical composition which comprises an HMG Co-A reductase inhibitor, or a pharmaceutically acceptable salt, solvate, solvate of such a salt or a prodrug thereof, in association with a pharmaceutically acceptable diluent or carrier for use in the treatment of hypercholesterolemia and/or other forms of dyslipidaemia wherein said hypercholesterolemia and dyslipidaemias are characterized by defects in lipoproteins or their receptors.

According to a further aspect of the invention there is provided a pharmaceutical composition which comprises an IBAT inhibitor, or a pharmaceutically acceptable salt, solvate, solvate of such a salt or a prodrug thereof, in association with a pharmaceutically acceptable diluent or carrier, in combination with a pharmaceutical composition which comprises an HMG Co-A reductase inhibitor, or a pharmaceutically acceptable salt, solvate, solvate of such a salt or a prodrug thereof, in association with a pharmaceutically acceptable diluent or carrier for use in lowering of abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

The pharmaceutical compositions may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository. In general the above compositions may be prepared in a conventional manner using conventional excipients.

According to a further aspect of the present invention there is provided a kit comprising an IBAT inhibitor, or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof; optionally with instructions for use; for use in the treatment of hypercholesterolemia and/or other forms of dyslipidaemia wherein said hypercholesterolemia and dyslipidaemias are characterized by defects in lipoproteins or their receptors.

According to a further aspect of the present invention there is provided a kit comprising an IBAT inhibitor, or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof; optionally with instructions for use; for use in lowering of abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

According to a further aspect of the present invention there is provided a kit comprising:
a) an IBAT inhibitor, or a pharmaceutically acceptable salt, solvate, solvate of such a salt or a prodrug thereof, in a first unit dosage form;
b) an HMG Co-A reductase inhibitor, or a pharmaceutically acceptable salt, solvate, solvate of such a salt or a prodrug thereof; in a second unit dosage form; and
c) container means for containing said first and second dosage forms; and optionally
d) with instructions for use;
   for use in the treatment of hypercholesterolemia and/or other forms of dyslipidaemia wherein said hypercholesterolemia and dyslipidaemias are characterized by defects in lipoproteins or their receptors.

According to a further aspect of the present invention there is provided a kit comprising:
a) an IBAT inhibitor, or a pharmaceutically acceptable salt, solvate, solvate of such a salt or a prodrug thereof, in a first unit dosage form;
b) an HMG Co-A reductase inhibitor, or a pharmaceutically acceptable salt, solvate, solvate of such a salt or a prodrug thereof; in a second unit dosage form; and
c) container means for containing said first and second dosage forms; and optionally
d) with instructions for use;
   for use in lowering of abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

According to a further aspect of the present invention there is provided a kit comprising:
a) an IBAT inhibitor, or a pharmaceutically acceptable salt, solvate, solvate of such a salt or a prodrug thereof, together with a pharmaceutically acceptable diluent or carrier, in a first unit dosage form;
b) an HMG Co-A reductase inhibitor, or a pharmaceutically acceptable salt, solvate, solvate of such a salt or a prodrug thereof, in a second unit dosage form; and
c) container means for containing said first and second dosage forms; and optionally
d) with instructions for use;
   for use in the treatment of hypercholesterolemia and/or other forms of dyslipidaemia wherein said hypercholesterolemia and dyslipidaemias are characterized by defects in lipoproteins or their receptors.

According to a further aspect of the present invention there is provided a kit comprising:
a) an IBAT inhibitor, or a pharmaceutically acceptable salt, solvate, solvate of such a salt or a prodrug thereof, together with a pharmaceutically acceptable diluent or carrier, in a first unit dosage form;
b) an HMG Co-A reductase inhibitor, or a pharmaceutically acceptable salt, solvate, solvate of such a salt or a prodrug thereof, in a second unit dosage form; and
c) container means for containing said first and second dosage forms; and optionally
d) with instructions for use;
   for use in lowering of abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

According to another feature of the invention there is provided the use of an IBAT inhibitor, or a pharmaceutically acceptable salt, solvate, solvate of such a salt or a prodrug thereof, in the manufacture of a medicament for use in the treatment of hypercholesterolemia and/or other forms of dyslipidaemia wherein said hypercholesterolemia and dyslipidaemias are characterized by defects in lipoproteins or their receptors, in a warm-blooded animal, such as man.

According to another feature of the invention there is provided the use of an IBAT inhibitor, or a pharmaceutically acceptable salt, solvate, solvate of such a salt or a prodrug thereof, in the manufacture of a medicament for use in lowering of abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

According to another feature of the invention there is provided the use of an IBAT inhibitor, or a pharmaceutically acceptable salt, solvate, solvate of such a salt or a prodrug thereof, in combination with an HMG Co-A reductase inhibitor, or a pharmaceutically acceptable salt, solvate, solvate of such a salt or a prodrug thereof, in the manufacture of a medicament for use in the treatment of hypercholesterolemia and/or other forms of dyslipidaemia wherein said hypercholesterolemia and dyslipidaemias are characterized by defects in lipoproteins or their receptors, in a warm-blooded animal, such as man.

According to another feature of the invention there is provided the use of an IBAT inhibitor, or a pharmaceutically acceptable salt, solvate, solvate of such a salt or a prodrug thereof, in combination with an HMG Co-A reductase inhibitor, or a pharmaceutically acceptable salt, solvate, solvate of such a salt or a prodrug thereof, in the manufacture of a medicament for use in lowering of abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects-in lipoproteins or their receptors.

According to another feature of the invention there is provided the use of an IBAT inhibitor, or a pharmaceutically acceptable salt, solvate, solvate of such a salt or a prodrug thereof, in the treatment of hypercholesterolemia and/or other forms of dyslipidaemia wherein said hypercholesterolemia and dyslipidaemias are characterized by defects in lipoproteins or their receptors, in a warm-blooded animal, such as man.

According to another feature of the invention there is provided the use of an IBAT inhibitor, or a pharmaceutically acceptable salt, solvate, solvate of such a salt or a prodrug thereof, in lowering of abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

According to another feature of the invention there is provided the use of an IBAT inhibitor, or a pharmaceutically acceptable salt, solvate, solvate of such a salt or a prodrug thereof, in combination with an HMG Co-A reductase inhibitor, or a pharmaceutically acceptable salt, solvate, solvate of such a salt or a prodrug thereof, in the treatment of hypercholesterolemia and/or other forms of dyslipidaemia wherein said hypercholesterolemia and dyslipidaemias are characterized by defects in lipoproteins or their receptors, in a warm-blooded animal, such as man.

According to another feature of the invention there is provided the use of an IBAT inhibitor, or a pharmaceutically acceptable salt, solvate, solvate of such a salt or a prodrug thereof, in combination with an HMG Co-A reductase inhibitor, or a pharmaceutically acceptable salt, solvate, solvate of such a salt or a prodrug thereof, in lowering abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

According to a further aspect of the present invention there is provided a combination comprising an IBAT inhibitor, or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, for use in the treatment of hypercholesterolemia and/or other forms of dyslipidaemia wherein said hypercholesterolemia and dyslipidaemias are characterized by defects in lipoproteins or their receptors.

According to a further aspect of the present invention there is provided a combination comprising an IBAT inhibitor, or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, for use in lowering abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

According to a further aspect of the present invention there is provided a combination treatment comprising the administration of an effective amount of an IBAT inhibitor, or a pharmaceutically acceptable salt, solvate, solvate of such a salt or a prodrug thereof, optionally together with a pharmaceutically acceptable diluent or carrier, in combination with an effective amount of an HMG Co-A reductase inhibitor, or a pharmaceutically acceptable salt, solvate, solvate of such a salt or a prodrug thereof, optionally together with a pharmaceutically acceptable diluent or carrier to a warm-blooded animal, such as man in need of such therapeutic treatment for use in the treatment of hypercholesterolemia and/or other forms of dyslipidaemia wherein said hypercholesterolemia and dyslipidaemias are characterized by defects in lipoproteins or their receptors.

According to a further aspect of the present invention there is provided a combination treatment comprising the administration of an effective amount of an IBAT inhibitor, or a pharmaceutically acceptable salt, solvate, solvate of such a salt or a prodrug thereof, optionally together with a pharmaceutically acceptable diluent or carrier, in combination with an effective amount of an HMG Co-A reductase inhibitor, or a pharmaceutically acceptable salt, solvate, solvate of such a salt or a prodrug thereof, optionally together with a pharmaceutically acceptable diluent or carrier to a warm-blooded animal, such as man in need of such therapeutic treatment for use in lowering abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

The IBAT inhibitor, or a pharmaceutically acceptable salt, solvate, solvate of such a salt or a prodrug thereof, will normally be administered to a warm-blooded animal at a unit dose within the range 5-5000 mg per square meter body area of the animal, i.e. approximately 0.01-50 mg/kg, and this would be expected to provide a therapeutically-effective dose. A unit dose from such as a tablet or capsule will usually contain, for example 1-250 mg of active ingredient. In one aspect of the invention a daily dose in the range of 0.02-50 mg/kg is employed. In another aspect a daily dose in the rage of 0.02-20 mg/kg is employed. However the daily dose will necessarily be varied depending upon the host treated, the particular route of administration, and the severity of the illness being treated. Accordingly the optimum dosage may be determined by the practitioner who is treating any particular patient.

The HMG CoA reductase inhibitor, or a pharmaceutically acceptable salt, solvate, solvate of such a salt or a prodrug thereof, will normally be administered to a warm-blooded animal at a unit dose within the range 0.5-100 mg per day, and this would be expected to provide a therapeutically-effective dose. In one aspect of the invention a daily dose in the range of 10-80 mg per day is employed. In another aspect a daily dose in the rage of 10-20 mg per day is employed. However the daily dose will necessarily be varied depending upon the host treated, the particular route of administration, and the severity of the illness being treated. Accordingly the optimum dosage may be determined by the practitioner who is treating any particular patient.

The dosage of each of the two drugs and their proportions have to be composed so that the best possible treatment effects, as defined by national and international guidelines (which are periodically reviewed and re-defined), will be met.

1. A method of treating hypercholesterolemia and/or other forms of dyslipidaemia wherein said hypercholesterolemia and dyslipidaemias are characterized by defects in lipoproteins or their receptors, in a warm-blooded animal, such as man, in need of such treatment which comprises administering to said animal an effective amount of an IBAT inhibitor, or a pharmaceutically acceptable salt, solvate, solvate of such a salt or a prodrug thereof.

2. A method of treating hypercholesterolemia and/or other forms of dyslipidaemia wherein said hypercholesterolemia and dyslipidaemias are characterized by defects in lipoproteins or their receptors, in a warm-blooded animal, such as man, in need of such treatment which comprises administering to said animal an effective amount of an IBAT inhibitor, or a pharmaceutically acceptable salt, solvate, solvate of such a salt or a prodrug thereof in combination with an effective amount of an HMG Co-A reductase inhibitor, or a pharmaceutically acceptable salt, solvate, solvate of such a salt or a prodrug thereof.

3. A pharmaceutical composition which comprises an IBAT inhibitor, or a pharmaceutically acceptable salt, solvate, solvate of such a salt or a prodrug thereof, in association with a pharmaceutically acceptable diluent or carrier for use in the treatment of hypercholesterolemia and/or other forms of dyslipidaemia wherein said hypercholesterolemia and dyslipidaemias are characterized by defects in lipoproteins or their receptors.

4. A pharmaceutical composition which comprises an IBAT inhibitor, or a pharmaceutically acceptable salt, solvate, solvate of such a salt or a prodrug thereof, in association with a pharmaceutically acceptable diluent or carrier, in combination with a pharmaceutical composition which comprises an HMG Co-A reductase inhibitor, or a pharmaceutically acceptable salt, solvate, solvate of such a salt or a prodrug thereof, in association with a pharmaceutically acceptable diluent or carrier for use in the treatment of hypercholesterolemia and/or other forms of dyslipidaemia wherein said hypercholesterolemia and dyslipidaemias are characterized by defects in lipoproteins or their receptors.

5. A pharmaceutical composition which comprises an IBAT inhibitor, or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, in association with a pharmaceutically acceptable diluent or carrier for use in the treatment of hypercholesterolemia and/or other forms of dyslipidaemia wherein said hypercholesterolemia and dyslipidaemias are characterized by defects in lipoproteins or their receptors.

6. The use of an IBAT inhibitor, or a pharmaceutically acceptable salt, solvate, solvate of such a salt or a prodrug thereof, in the manufacture of a medicament for use in the treatment of hypercholesterolemia and/or other forms of dyslipidaemia wherein said hypercholesterolemia and dyslipidaemias are characterized by defects in lipoproteins or their receptors, in a warm-blooded animal, such as man.

7. The use of an IBAT inhibitor, or a pharmaceutically acceptable salt, solvate, solvate of such a salt or a prodrug thereof, in combination with an HMG Co-A reductase inhibitor, or a pharmaceutically acceptable salt, solvate, solvate of such a salt or a prodrug thereof, in the manufacture of a medicament for use in the treatment of hypercholesterolemia and/or other forms of dyslipidaemia wherein said hypercholesterolemia and dyslipidaemias are characterized by defects in lipoproteins or their receptors, in a warm-blooded animal, such as man.

8. The use of an IBAT inhibitor, or a pharmaceutically acceptable salt, solvate, solvate of such a salt or a prodrug thereof, in the treatment of hypercholesterolemia and/or other forms of dyslipidaemia wherein said hypercholesterolemia and dyslipidaemias are characterized by defects in lipoproteins or their receptors, in a warm-blooded animal, such as man.

9. The use of an IBAT inhibitor, or a pharmaceutically acceptable salt, solvate, solvate of such a salt or a prodrug thereof, in combination with an HMG Co-A reductase inhibitor, or a pharmaceutically acceptable salt, solvate, solvate of such a salt or a prodrug thereof, in the treatment of hypercholesterolemia and/or other forms of dyslipidaemia wherein said hypercholesterolemia and dyslipidaemias are characterized by defects in lipoproteins or their receptors, in a warm-blooded animal, such as man.

10. A method of testing whether an IBAT inhibitor, or a pharmaceutically acceptable salt, solvate, solvate of such a salt or a prodrug thereof has any one of the following effects:
i) lowering total cholesterol; optionally in combination with an HMG CoA reductase inhibitor, or a pharmaceutically acceptable salt, solvate, solvate of such a salt or a prodrug thereof;
ii) lowering LP-remnants; optionally in combination with an HMG CoA reductase inhibitor, or a pharmaceutically acceptable salt, solvate, solvate of such a salt or a prodrug thereof;
iii) lowering LDL; optionally in combination with an HMG CoA reductase inhibitor, or a pharmaceutically acceptable salt, solvate, solvate of such a salt or a prodrug thereof;
iv) raising HDL; optionally in combination with an HMG CoA reductase inhibitor, or a pharmaceutically acceptable salt, solvate, solvate of such a salt or a prodrug thereof; or
v) exhibits a synergistic effect in combination with an HMG CoA reductase inhibitor, or a pharmaceutically acceptable salt, solvate, solvate of such a salt or a prodrug thereof on the lowering of the ratio of (LP-remnants+LDL-cholesterol)/(HDL-cholesterol);
wherein the method of testing comprises administering the IBAT inhibitor, or a pharmaceutically acceptable salt, solvate, solvate of such a salt or a prodrug thereof, to a transgenic LDL receptor and/or ApoE deficient non-human mammal optionally in combination with an HMG CoA reductase inhibitor, or a pharmaceutically acceptable salt, solvate, solvate of such a salt or a prodrug thereof; and determining whether there has been an effect on any one of (i)-(v) above on the non human mammal.

11. A combination comprising an IBAT inhibitor, or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, for use in the treatment of hypercholesterolemia and/or other forms of dyslipidaemia wherein said hypercholesterolemia and dyslipidaemias are characterized by defects in lipoproteins or their receptors.

12. A combination comprising an IBAT inhibitor, or a pharmaceutically acceptable salt, solvate, solvate of such a salt or a prodrug thereof, and an HMG Co-A reductase inhibitor, or a pharmaceutically acceptable salt, solvate, solvate of such a salt or a prodrug thereof, for use in lowering abnormal cholesterol and triglyceride levels and the composition of the different lipoproteins concerning cholesterol, triglycerides, and apolipoproteins in a warm-blooded animal, such as man, with hypercholesterolemia and/or other forms of dyslipidaemia wherein said hypercholesterolemia and dyslipidaemias are characterized by defects in lipoproteins or their receptors.

13. The method of treatment, pharmaceutical composition, use, method of testing or combination according to any one of claims 1-12 wherein the IBAT inhibitor is (3R,5R)-3-butyl-3-ethyl-1,1-dioxido-5-phenyl-2,3,4,5-tetrahydro-1,4-benzothiazepin-8-yl β-D-glucopyranosiduronic acid or a pharmaceutically acceptable salt, solvate, solvate of such a salt or a prodrug thereof.

14. The method of treatment, pharmaceutical composition, use, method of testing or combination according to any one of claims 1-12 wherein the IBAT inhibitor is selected from:
1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(carboxymethyl)carbamoyl]4 hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-1 ‘-phenyl-1 ’-[N′-(2-sulphoethyl)carbamoyl]methyl}carbamoylmethoxy)-2,3,4,5-tetrahydro-1,5-benzothiazepine;
1,1-dioxo-3-butyl-3-ethyl-5-phenyl-7-methylthio-8-(N-{R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-sulphoethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-sulphoethyl) carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(5-carboxypentyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{α-[N′-(2-sulphoethyl)carbamoyl]-2-fluorobenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N-[(R)-α-(N′-{(R)-1-[N″-(R)-(2-hydroxy-1-carboxyethyl)carbamoyl]-2-hydroxyethyl}carbamoyl)benzyl]carbamoylmethoxy}-2,3,4,5-tetrahydro-1,5-benzothiazepine;
1,1-dioxo-3-butyl-3-ethyl-5-phenyl-7-methylthio-8-(N-{α-[N-(carboxymethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{α-[N′-((ethoxy)(methyl)phosphoryl-methyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-{N-[(R)-α-(N′-{2-[(hydroxy)(methyl)phosphoryl]ethyl}carbamoyl)benzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[N′-(2-methylthio-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N-[(R)-α-(N′-{2-[(methyl)(ethyl) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-{N-[(R)-α-(N′-{2-[(methyl)(hydroxy) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[(R)—N′-(2-methylsulphinyl-1-carboxyethyl)carbamoyl]benzyl}carbamoylmethoxy)-2,3,4,5-tetrahydro-1,5-benzothiazepine; and
1,1-dioxo-3,3-dibutyl-5-phenyl-7-methoxy-8-[N-{(R)-α-[N′-(2-sulphoethyl)carbamoyl]-4-hydroxybenzyl}carbamoylmethoxy]-2,3,4,5-tetrahydro-1,5-benzothiazepine;
or a pharmaceutically acceptable salt, solvate, solvate of such a salt or a prodrug thereof.

15. The method of treatment, pharmaceutical composition, use, method of testing or combination according to any one of claims 2, 4, 5, 7, 9, 10, 11, 12, 13 or 14 wherein the HMG CoA reductase inhibitor is selected from fluvastatin, lovastatin, pravastatin, simvastatin, atorvastatin, cerivastatin, bervastatin, dalvastatin, mevastatin and rosuvastatin, or a pharmaceutically acceptable salt, solvate, solvate of such a salt or a prodrug thereof.

16. The method of treatment, pharmaceutical composition, use, method of testing or combination according to any one of claims 2, 4, 5, 7, 9, 10, 11, 12, 13, 14 or 15 wherein the HMG CoA reductase inhibitor is atorvastatin calcium salt.

17. The method of treatment, pharmaceutical composition, use, method of testing or combination according to any one of claims 2, 4, 5, 7, 9, 10, 11, 12, 13, 14 or 15 wherein the HMG CoA reductase inhibitor is rosuvastatin calcium salt.

18. The method of treatment, pharmaceutical composition, use, method of testing or combination according to any one of claims 1-17 wherein “hypercholesterolemia and/or other forms of dyslipidaemia wherein said hypercholesterolemia and dyslipidaemias are characterized by defects in lipoproteins or their receptors” is the disease state familial hypercholesterolemia.

19. The method of treatment, pharmaceutical composition, use, method of testing or combination according to any one of claims 1-17 wherein “hypercholesterolemia and/or other forms of dyslipidaemia wherein said hypercholesterolemia and dyslipidaemias are characterized by defects in lipoproteins, or their receptors” is the disease state familial defective apolipoprotein B 100.

20. The method of treatment, pharmaceutical composition, use, method of testing or combination according to any one of claims 1-17 wherein “hypercholesterolemia and/or other forms of dyslipidaemia wherein said hypercholesterolemia and dyslipidaemias are characterized by defects in lipoproteins or their receptors” is the disease state type III dyslipidaemia.
